(19) United States
(12) Patent Application Publication
(10) Pub. No.: US 2026/0283464 A1
(43) Pub. Date: Sep. 24, 2026

(54) WIRELESS PHYSIOLOGICAL SENSOR CONNECTIVITY FOR PATIENT MONITOR

(71) Applicant: DRÄGERWERK AG & CO. KGAA, Lübeck (DE)

(72) Inventors: Vinphin Inasu KUNDUKULAM VARGHESE, Windham, NH (US); Georgios K. KOKOVIDIS, Waltham, MA (US)

(21) Appl. No.: 19/472,559

(22) PCT Filed: Apr. 5, 2024
(86) PCT No.: PCT/IB2024/053376

§ 371 Date: Oct. 3, 2025

Related U.S. Application Data
(60) Provisional application No. 63/457,875, filed on Apr. 7, 2023.

(51) Int. Cl. A61B 5/00

(52) U.S. Cl. CPC A61B 5/0002; A61B 2560/045; A61B 2562/227

(57) ABSTRACT

A system and method for enabling patient monitors (that do not have built in wireless capability) to connect to wireless physiological sensors. The patient monitor is equipped with connector ports arranged for communicating with patient physiological sensors through wired connections, such as cables. A wireless interface device includes a connector and is arranged for insertion into one of the connector ports otherwise intended for receiving wired sensors. Using a wireless communications protocol, the wireless interface device communicates with one or more wireless sensors attached to the body of a patient, the wireless sensors transmitting data signals indicative of physiological parameters of the patient. The wireless interface receives the data signals from the wireless sensors and transmits them to the patient monitor. A processor processes the received sensor data signals to enable recognition by the patient monitor.

### BACKGROUND

Patient monitors are configured to receive physiological data from another device, such as one or more sensors, and either display a patient's physiological data, monitor a patient's physiological data, or both. Such patient monitors may be configured to be worn by a patient, may be a hand-held device, may be docked to, or undocked from a larger unit such as a monitor mount, and, thus, may be transportable. Many existing patient monitors require attachment of one or more sensors to a patient in order to detect physiological signals from the patient. Signals detected by the sensors are transmitted from the sensors to the monitor through a cable or wire connected at one end to the sensor and at the other end to a wired input, e.g., a connector, located in the patient monitor. The patient monitor processes the received signals and determines vital signs such as the patient's pulse rate, respiration rate (RR), and arterial oxygen saturation (SpO2). For example, a pulse oximeter is a finger sensor that can include two light emitters and a photodetector. The sensor emits light into the patient's finger and transmits the detected light signal to the monitor through a cable physically and electrically connected to a wired input on the patient monitor. The patient monitor includes a processor that processes the signal, determines vital signs (e.g., pulse rate, respiration rate, arterial oxygen saturation SpO2), and displays the vital signs on a display. Such monitors can detect other physiological data through other types of sensors such as electrocardiogram (ECG) sensors, body temperature (BT) sensors, non-invasive blood pressure (NIBP) cuffs, invasive blood pressure sensors, end-tidal carbon dioxide (etCO2) sensors, and breathing rate (BR) sensors, to name a few. However, the patient monitor has a limited number of wired inputs, e.g., four fixed connector ports, so there is a limit as to the number of sensor cables that can be attached and the types of physiological data that can be monitored.

Also, where a patient is connected to such sensor wires and cables, during transport, or when the patient is turned, these sensor cables can become entangled, possibly resulting in damage to the cables, or injury to the patient. Where critical vital signs need to be continuously monitored, sensor cables can become disconnected from the legacy wired patient monitor, from the patient, or from both. Managing sensor cables in a hospital environment can be a time consuming task. This has led to the development of wireless physiological sensors and patient monitors capable of interfacing with wireless sensors.

Having the option to use wireless sensors with patient monitors is desirable in many medical environments but providers may be reluctant to replace existing patient monitors (without wireless sensor connectivity) due to replacement cost and the fact that the existing patient monitor may have significant remaining service life. Therefore, there is a need for a solution that allows existing patient monitors to be used with wireless physiological sensors.

Under the present invention, wireless functionality is provided to such patient monitors arranged to accept only a limited number of wired sensor devices. In particular, the system is provided with a wireless interface device arranged to be physically and electrically connected to the patient monitor. The wireless interface device utilizes a wireless communication protocol to receive sensor data signals from one or more wireless sensor devices attached to the body of a patient. The wireless interface device then processes the received sensor data signals and transmits them to the patient monitor. Providing such wireless functionality expands the number and type of parameters that can be monitored simultaneously by the patient monitor and increases the utility of the connector ports. Also, providing wireless functionality eliminates the disadvantages and hazards associated with cables and wires discussed above.

### SUMMARY

In the exemplary implementations described herein, a system and method provide wireless functionality to a patient monitor, to enable use of the patient monitor with wireless physiological sensors. The system comprises a patient monitor for monitoring various physiological parameters of a patient, such as end-tidal carbon dioxide, oxygen saturation, and body temperature. Patient monitor is herein defined as a physiological patient monitor or other device configured to receive physiological data and/or signals from a patient including a ventilator, a workstation, or the like. The patient monitor does not have built-in wireless physiological sensor capability, and includes a limited number of connector ports arranged to receive data signals from wired sensors over cables physically connected to the patient monitor. A wireless interface device is adapted to physically and electrically connect to one or more of the existing connector ports of the patient monitor. The wireless interface device receives power from the existing connector port, communicates using a wireless communication protocol with one or more physiological sensors, and transmits data received from the physiological sensors to the patient monitor. The firmware of the patient monitor is updated to communicate with the wireless interface device and to properly interpret the physiological sensor data received through the wireless interface device.

The following are additional aspects of the invention.

Aspect 1: A method of enabling a patient monitor to establish a data connection with at least one wireless physiological sensor through at least one wired physiological sensor connector port of the patient monitor, the method comprising:
providing a wireless interface device having a wireless communication interface and at least one wireless interface device connector, the wireless interface device being adapted to establish a wireless data connection with at least one wireless physiological sensor and to mechanically and electrically connect to the at least one wired physiological sensor connector port of the patient monitor through the at least one wireless interface device connector; and
reprogramming the patient monitor (a) to enable the patient monitor to detect a wireless interface device connected to the patient monitor through one of the at least one wired physiological sensor connector port and (b) to enable the patient monitor to pair to the detected wireless interface device.

Aspect 2: The method of aspect 1, wherein the reprogramming step further comprises reprogramming the patient monitor to (c) detect a wireless communication connection between the wireless interface device and at least one wireless physiological sensor.

Aspect 3: The method of aspect 2, wherein the reprogramming step further comprises reprogramming the patient monitor (d) to enable identification of a wireless sensor type for each detected wireless communication connection.

Aspect 4: The method of aspect 3, wherein the reprogramming step further comprises reprogramming the patient monitor (d) to enable identification of a wireless sensor type for each detected wireless communication connection, wherein identification of a wireless sensor type comprises enabling a user to select the wireless sensor type using a menu displayed by the patient monitor.

Aspect 5: The method of any one of aspects 3-4, wherein the reprogramming step further comprises reprogramming the patient monitor (d) to enable identification of a wireless sensor type for each detected wireless communication connection based on data received by the patient monitor from each at least one wireless physiological sensor.

Aspect 6: The method of any one of aspects 2-5, wherein the reprogramming step further comprises reprogramming the patient monitor to enable the display of data received by the patient monitor from each of the at least one wireless physiological sensor.

Aspect 7: The method of any one of aspects 2-6, wherein each at least one wired physiological sensor connector port is configured to receive a wired physiological sensor that is adapted to gather data representative of a first physiological parameter;
wherein the reprogramming step further comprises reprogramming the patient monitor to (f) enable the display of data received by the patient monitor from a one wireless physiological sensor adapted to gather data representative of a second physiological parameter that is different from the first physiological parameter.

Aspect 8: The method of aspect 7, wherein each of the first and second physiological parameters is selected from the group of: end-tidal carbon dioxide (EtCO2), pulse oximetry (SpO2), electrocardiogram (ECG), electroencephalography (“EEG”), pulse, heart rate (HR), respiratory rate (RR), apnea detection, blood pressure, and body temperature (BT).

Aspect 9: A wireless interface device for use with a patient monitor having at least one wired physiological sensor port, each at least one wired physiological sensor connector port having a plurality of physiological sensor port contacts, the wireless interface device comprising:
at least one connector, each at least one connector comprising a plurality of electrical contacts including a plurality of data I/O contacts and a plurality of power contacts.
a power module in electrical communication with the plurality of power contacts; and
a wireless communication module in electrical contact with the data I/O contacts;
wherein the at least one connector is shaped to be physically connected to one of the at least one wired physiological sensor ports and to enable each of the plurality of connector electrical contacts to be electrically connected with one of the plurality of physiological sensor port contacts when the at least one connector is physically connected to the at least one wired physiological sensor port;
wherein the wireless communication module is adapted to establish a wireless data connection with at least one wireless physiological sensor; and
wherein when the at least one connector is connected to one of the at least one wired physiological sensor port, the wireless communication module is adapted to pair with the patient monitor and transmit received data from the at least one wireless physiological sensor through at least one of the at least one wired physiological sensor port.

Aspect 10: The wireless interface device of aspect 9, wherein the power module further comprises a voltage regulator that limits a voltage output of the power module to a predetermined voltage.

Aspect 11: The wireless interface device of aspect 10, wherein the predetermined voltage is 3.3 volts.

Aspect 12: The wireless interface of any one of aspects 9-11, wherein the at least one wireless physiological sensor comprises a plurality of wireless physiological sensors.

Aspect 13: The wireless interface of any one of aspects 9-12, wherein the wireless communication module is adapted to communicate using a wireless communication protocol selected from the group of: Bluetooth, ZigBee, thread, infrared, ANT, RF, and Wi-Fi.

Aspect 14: The wireless interface device of any one of aspects 9-13, wherein the at least one wired physiological sensor port comprises a plurality of wired physiological sensor ports, the at least one connector of the wireless interface device comprises a plurality of connectors, and the plurality of connectors being equal or less in number than the plurality of wired physiological sensor ports.

Aspect 15: The wireless interface device of aspect 14, wherein each of the plurality of connectors is adapted the mechanically and electrically engage a respective one of the plurality of wired physiological sensor ports.

Aspect 16: The wireless interface device of any one of aspects 14-15, wherein each of the plurality of connectors is keyed to engage a respective one of the plurality of wired physiological sensor ports.

Aspect 17: The wireless interface device of any one of aspects 14-16, further comprising a housing to which the plurality of connectors, the power module, and the wireless communication module are attached, wherein the housing is adapted to removably engage a patient monitor.

Aspect 18: The wireless interface device of aspect 17, wherein the housing is c-shaped.

Aspect 19: The wireless interface device of any one of aspects 17-18, wherein the housing is adapted allow a power port of a patient monitor to remain exposed when each of the plurality of connectors is mechanically and electrically engaged to a respective one of the plurality of wired physiological sensor ports.

Aspect 20: The wireless interface device of aspect 19, wherein the housing comprises a serration that enables a first portion of the housing covering to be separated from the housing, wherein the first portion covers the power port when each of the plurality of connectors is mechanically and electrically engaged to a respective one of the plurality of wired physiological sensor ports.

Aspect 21: The wireless interface device of any one of aspects 19-20, further comprising first wired sensor interface connector adapted to establish a wired data connection with a wired physiological sensor.

Aspect 22: A patient monitor comprising:
at least one processor, memory, a wireless communication interface adapted to communicate with a network, a graphical user interface, a power source, and a plurality of sensor ports, the plurality of sensor ports comprising a first sensor port;
wherein the at least one processor is adapted to detect whether a wired physiological sensor or a wireless interface device is connected to the first sensor port, the wired physiological sensor being adapted to sense a first physiological parameter, the wireless interface device being adapted to connect with a first wireless physiological sensor adapted to sense a second physiological parameter;
wherein when the wired physiological sensor is detected as being connected to the first sensor port, the at least one processor is adapted to display on the graphical user interface data representative of the physiological parameter based on data received from the wired physiological sensor; and
wherein when the wireless interface device is detected as being connected to the first sensor port, the at least one processor is adapted to display on the graphical user interface data representative of the second physiological parameter based on data received from a wireless the wireless physiological sensor.

Aspect 23: The patient monitor of aspect 22, wherein the second physiological parameter is different from the first physiological parameter.

Aspect 24: The patient monitor of any one of aspects 22-23, wherein the processor further is adapted to detect a wireless communication connection between the wireless interface device and at least one wireless physiological sensor.

Aspect 25: The patient monitor of aspect 24, wherein the processor further is adapted to identify a wireless sensor type for each detected wireless communication connection.

Aspect 26: The patient monitor of any one of aspects 24-25, wherein the processor is further adapted to identify a wireless sensor type for each detected wireless communication connection based on user input using a menu displayed by the patient monitor.

Aspect 27: The patient monitor of any one of aspects 24-26, wherein the processor is further adapted to identify a wireless sensor type for each detected wireless communication connection based on data received by the patient monitor from each at least one wireless physiological sensor.

Aspect 28: The patient monitor of any one of aspects 22-27, wherein each of the first and second physiological parameters is selected from the group of: end-tidal carbon dioxide (EtCO2), pulse oximetry (SpO2), electrocardiogram (ECG), electroencephalography (“EEG”), pulse, heart rate (HR), respiratory rate (RR), apnea detection, and blood pressure.

Aspect 29: A method of enabling a patient monitor to establish a data connection with at least one wireless physiological sensor through a plurality of wired physiological sensor connector ports of the patient monitor, the method comprising:
providing a wireless interface device having a housing that contains a wireless communication interface, a power module, and a plurality of wireless interface device connectors, the wireless interface device being adapted to establish a wireless data connection with at least one wireless physiological sensor, the plurality of wireless interface device connectors each being arranged on the housing to enable connection with a respective one of the plurality of wired physiological sensor connector ports;
mechanically and electrically connecting each of the plurality of wired physiological sensor connector ports with a respective one of the plurality of wireless interface device connectors; receiving physiological data from at least one wireless sensor to the wireless communication interface; and
transmitting the received physiological data to the patient monitor through at least one of the plurality of wired physiological sensor connector ports.

Aspect 30: The method of aspect 29, wherein each of the plurality of wireless interface device connectors is keyed to enable connection with a respective one of the plurality of wired physiological sensor connector ports.

Aspect 31: The method of any one of aspects 29-30, further comprising removably engaging the patient monitor with the housing.

Aspect 32: The method of any one of aspects 29-31, further comprising leaving a power port of the patient monitor exposed and able to be connected to a power cable when each of the plurality of wired physiological sensor connector ports is mechanically and electrically connected with the respective one of the plurality of wireless interface device connectors.

Aspect 33: The method of any one of aspects 29-32, further comprising breaking away a first portion of the housing along a serration to expose a power port when each of the plurality of connectors is mechanically and electrically engaged to a respective one of the plurality of wired physiological sensor ports.

Aspect 34: A method of enabling a patient monitor to establish a data connection with a plurality of physiological sensors through a first wired physiological sensor connector port of the patient monitor, the first wired physiological sensor connector port being configured to receive a wired physiological sensor that is adapted to gather data representative of a first physiological parameter, the method comprising:
(a) providing a sensor interface device having a wireless communication interface, a first wired sensor interface connector, and a patient monitor connector, the sensor interface device being adapted to (i) mechanically and electrically connect to the first wired physiological sensor connector port through the patient monitor connector, (ii) establish a wireless data connection with at least one wireless physiological sensor, and (iii) establish a wired data connection with a first wired physiological sensor through the first wired sensor interface connector; and
(b) reprogramming the patient monitor to (i) enable the patient monitor to detect a wireless interface device connected to the patient monitor through one of the at least one wired physiological sensor connector port, (ii) enable the patient monitor to pair to the detected wireless interface device, (iii) to receive data from the at least one wireless physiological sensor that has established a connection with the sensor interface, and (iv) to receive data from the first wired physiological sensor when the first physiological sensor is connected to the first wired sensor interface connector.

Aspect 35: The method of aspect 34, wherein the at least one wireless physiological sensor comprises a plurality of wireless physiological sensors.

Aspect 36: The method of aspect 35, wherein the plurality of wireless physiological sensors comprise a first wireless physiological sensor adapted to provide a signal representative of a first physiological parameter and a second wireless physiological sensor adapted to provide a signal representative of a second physiological parameter, the first physiological parameter being different from the second physiological parameter.

Aspect 37: The method of aspect 36, wherein the first physiological parameter and the second physiological parameter are each selected from the group of: end-tidal carbon dioxide (EtCO2), pulse oximetry (SpO2), electrocardiogram (ECG), electroencephalography (“EEG”), pulse, heart rate (HR), respiratory rate (RR), apnea detection, and blood pressure.

Aspect 38: An interface device for a first physiological sensor port of a patient monitor, the first physiological sensor port being adapted to receive data representative of a first physiological parameter from a wired physiological sensor adapter, the interface comprising:
a patient monitor interface connector adapted to electrically and mechanically connect with the first physiological sensor port; and
a physiological sensor interface port adapted to electrically and mechanically connect with a wired physiological sensor adapted to provide a signal representative of a second physiological parameter, the second physiological parameter being different from the first physiological parameter.

Aspect 39: The interface device of aspect 38, wherein the first physiological parameter and the second physiological parameter are each selected from the group of first physiological parameter is selected from the group of: end-tidal carbon dioxide (EtCO2), pulse oximetry (SpO2), electrocardiogram (ECG), electroencephalography (“EEG”), pulse, heart rate (HR), respiratory rate (RR), apnea detection, and blood pressure.

Aspect 40: The interface device of any one of aspects 38-39, wherein the physiological sensor interface port is keyed differently from the first physiological sensor port.

Aspect 41: The interface device of any one of aspects 38-40, wherein the physiological sensor interface port is keyed identically to a second physiological sensor port of the patient monitor.

Aspect 42: The interface device of aspect 41, wherein the second physiological port is adapted to receive a signal representative of the second physiological parameter.

Aspect 43: The interface device of any one of aspects 38-42, wherein the physiological sensor interface port comprises a plurality of electrical contacts including a plurality of data I/O contacts and a plurality of power contacts.

Aspect 44: a method comprising:
(a) providing a patient monitor having a first physiological sensor port adapted to receive data representative of a first physiological parameter from a wired physiological sensor adapter;
(b) providing a sensor interface having a patient monitor connector and a wire sensor interface connector adapted to receive data representative of a second physiological parameter, the second physiological parameter being different from the first physiological parameter; and
(c) reprogramming the patient monitor to enable the first physiological sensor port to receive data representative of the second physiological parameter.

Aspect 45: The method of aspect 44, wherein step (e) further comprises providing the sensor interface with a wireless communication interface adapted to establish a wireless data connection with at least one wireless physiological sensor.

Aspect 46: The method of aspect 45, wherein step (f) further comprises reprogramming the patient monitor to enable the first physiological sensor port to receive data representative of a plurality of physiological parameters.

Aspect 47: The method of aspect 46, wherein the plurality of physiological parameters comprise end-tidal carbon dioxide (EtCO2), pulse oximetry (SpO2), electrocardiogram (ECG), electroencephalography (“EEG”), pulse, heart rate (HR), respiratory rate (RR), apnea detection, and blood pressure.

### BRIEF DESCRIPTION OF THE DRAWINGS

The invention will be described in conjunction with the following drawings in which like reference numerals designate like elements and wherein:

FIG. 1 shows a patient physiological monitoring system according to one or more embodiments of the present invention;

FIG. 2 shows a perspective view of a patient monitor provided with a wireless interface device, e.g., a wireless dongle, physically and electrically connected to a connector port of the patient monitor to provide wireless capability;

FIG. 3 is a schematic diagram of the system for providing wireless functionality to a patient monitor, wherein the patient monitor is in wireless communication with a plurality of different types of wireless sensor devices;

FIG. 4 is a schematic diagram illustrating connector ports located at the front end of the patient monitor, each connector port having a plurality of inputs arranged in a pattern, and originally designed for receiving a wired input;

FIG. 5 shows a rear perspective view of another exemplary implementation of a wireless interface device; and,

FIG. 6 shows a front perspective view of the wireless interface device of FIG. 5;

FIG. 7 shows a right side view of the wireless interface device of FIG. 5;

FIG. 8 shows a perspective view of a patient monitor provided with an interface device physically and electrically connected to a connector port of the patient monitor to provide a type of wired sensor than the connector port to which the interface device is connected;

FIG. 9 is a schematic diagram of the system shown in FIG. 8;

FIG. 10 shows a perspective view of a patient monitor provided with an interface device physically and electrically connected to a connector port of the patient monitor to provide wired and wireless sensor connectivity; and

FIG. 11 is a schematic diagram of the patient monitor provided and interface device of FIG. 10.

### DETAILED DESCRIPTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign particular meaning. Further, it should be noted that, as recited in the specification and in the claims appended hereto, the singular forms “a,” “an,” and “the” include the plural referents unless otherwise stated. Additionally, the terms “comprises” and “comprising” when used herein specify that certain features are present in that embodiment, but should not be interpreted to preclude the presence or addition of additional features, components, operations, and/or groups thereof.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Directional terminology, such as “top”, “bottom”, “below”, “above”, “front”, “behind”, “back”, “leading”, “trailing”, etc., may be used with reference to the orientation of the figures being described. Because parts of embodiments can be positioned in a number of different orientations, the directional terminology is used for purposes of illustration. It is to be understood that other embodiments may be utilized, and structural or logical changes may be made without departing from the scope defined by the claims. The following detailed description, therefore, is not to be taken in a limiting sense. Directional terminology used in the claims may aid in defining one element's spatial or positional relation to another element or feature, without being limited to a specific orientation.

Instructions may be executed by one or more processors, such as one or more central processing units (CPU), digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

As used herein, the term “physiological parameter” means a parameter that is representative of operation of the human body. Examples include, but are not limited to, end-tidal carbon dioxide (etCO2), SCIO, patient gas, thermoregulation, blood pressure, heart related measurement, pulse oximetry, respiration, neonatal measurement, and ventilation. Similarly, a “physiological sensor” means a device that is designed to detect a signal that is representative of a physiological parameter.

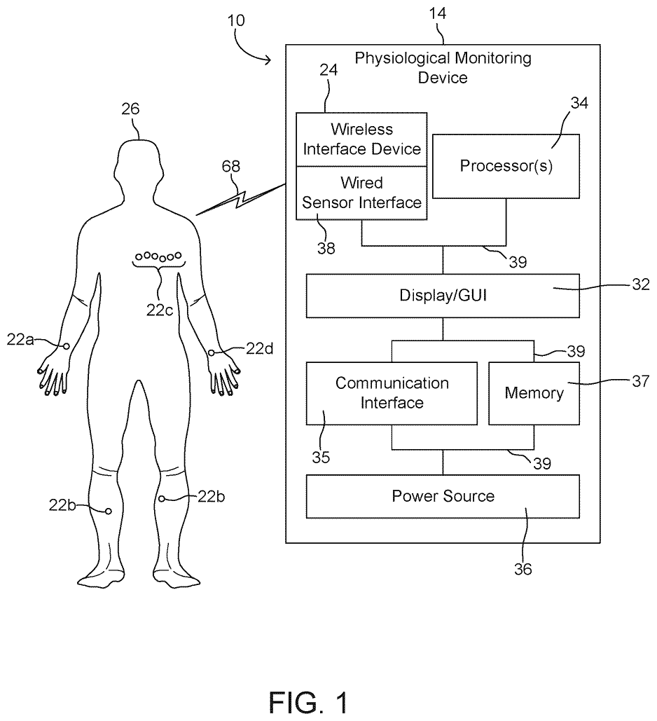

FIG. 1 shows a physiological monitoring system 10 according to one or more embodiments of the present invention. The physiological monitoring system 10 may include a physiological monitoring device. The physiological monitoring device is, for example, a patient monitor 14 implemented to monitor various physiological parameters of a patient 26 by way of a plurality of wired sensors (not shown) connected to the body of a patient. The patient monitor 14 includes a wired sensor interface 38 for physically and electrically connecting with one or more of the pluralities of wired sensors (not shown).

The wired sensors (not shown) may include sensors that are manufactured by different entities (e.g., companies), and sensors that are designed for different protocols or standards. The wired sensors (not shown) are arranged to be connected to a patient for sensing and transmitting data representative of various physiological parameters of the patient including but not limited to end-tidal carbon dioxide (EtCO2), various blood parameters, pulse oximetry (SpO2) signals, electrocardiogram (ECG) signals, electroencephalography (“EEG”) signals, blood pressure signals, and respiratory rate. Other physiological signals coupled via a wired interface could be transmitted wirelessly. The patient monitor 14 also includes one or more processors 34, a display/GUI 32, a communication interface 35, a memory 37, and a power source 36.

The communications interface 35 allows the patient monitor 14 to directly or indirectly (via, for example, the monitor mount (not shown) communicate with one or more computing networks and devices (not shown). The communications interface 35 can include various network cards, interfaces, or circuitry to enable wired and wireless communications with such computing networks and devices. The communications interface 35 can also enable direct device-to-device connection to other devices such as to a tablet, PC, or similar electronic device, or to an external storage device or memory.

The power source 36 can include a self-contained power source such as a battery pack and/or include an interface to be powered through an electrical outlet. The power source 36 can also be a rechargeable battery that can be detached allowing for replacement. In the case of a rechargeable battery, a small built-in backup battery (or super capacitor) can be provided for continuous power to be provided to the patient monitor 14 during battery replacement. Communication between the components of the patient monitor 14 (e.g., 32, 34, 36, 37, 38) are established using an internal bus 39. By itself, the patient monitor 14 is not provided with capability to communicate with wireless sensors. The patient monitor 14 may be configured to be worn by a patient, may be a hand-held device, may be docked to or undocked from a larger unit such as a monitor mount, and, thus, may be transportable.

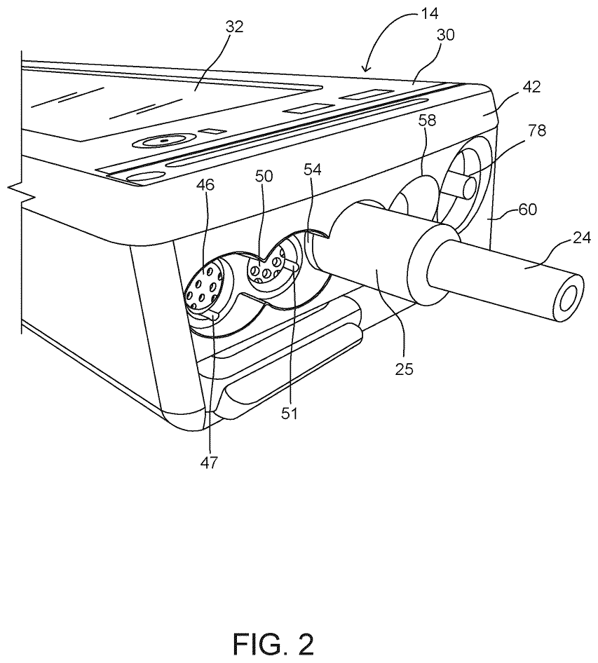

FIG. 2 shows a perspective view of the patient monitor 14 which includes a housing 30 having the display/GUI 32 on its front side and a docking interface (not shown) on its rear side. A plurality of connector ports 46, 50, 54, and 58 and a power port 78 are located at a connector end 60 of the housing 30. The power port 78 is adapted to be connected to a power cable (not shown) that is designed to enable the patient monitor 14 to be connected to an external power source. Each of the connector ports 46, 50, 54, and 58 is electrically connected to the wired sensor interface 38. The one or more connector ports 46, 50, 54, and 58 may be arranged to connect with connectors of the one or more varieties of wired sensors (not shown) for measuring and collecting different physiological parameters of the patient. Each type of wired sensor can include a specific type of cable connector for connection to another device, such as to the one or more of the connector ports 46, 50, 54, and 58 of the patient monitor 14. To allow multiple types of wired sensors to be connected to the patient monitor 14, the connector ports 46, 50, 54, and 58 can advantageously include a variety of connectors. However, due to its relatively small size, the patient monitor 14 can only provide a limited number of connector ports, e.g., four connector ports as shown in FIG. 2, for connecting to wired sensors, thus limiting the number and types of physiological information that can be gathered simultaneously.

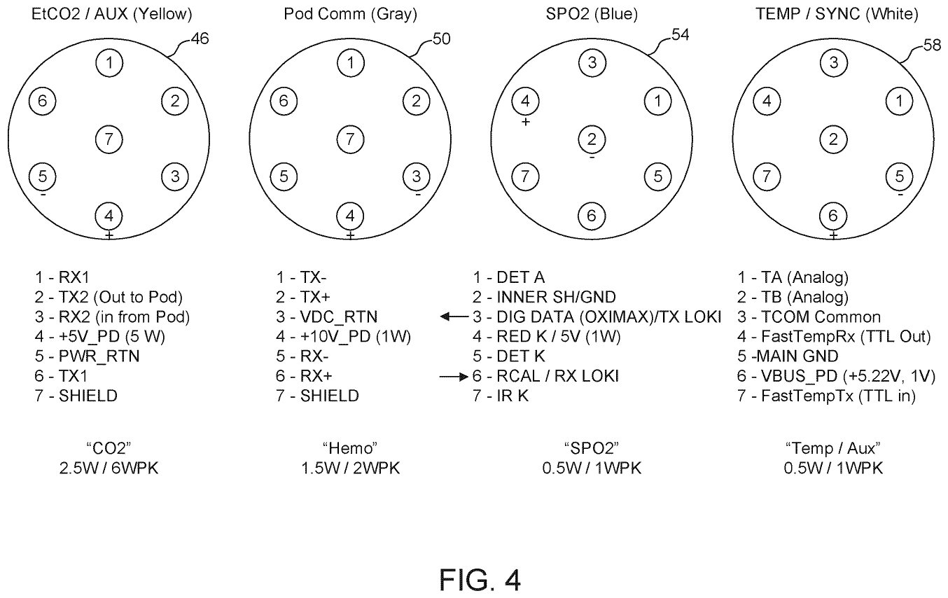

FIG. 4 illustrates example ports for receiving a wireless interface device. For example, as best shown in FIG. 4, the patient monitor 14 includes a connector port 46 labeled “CO2” provided for connecting with a wired end-tidal CO2 sensor (not shown); a port 50 labeled “hemo” provided for connecting with a wired sensor for measuring different blood parameters or analytes (not shown); a pulse oximetry port 54 labeled “SpO2” provided for connecting with a wired pulse oximetry SpO2 finger cuff (not shown); and, a port 58 labeled “Temp/Aux” provided for connecting with a wired body temperature probe (not shown). Under the patient monitor 14, other types of connector ports may be provided for connecting with other types of wired devices and/or sensors, e.g., an electrocardiogram device (ECG) for attaching to the chest of a patient, or a blood pressure monitor for attaching to the arm of a patient. It is contemplated that other types of connectors may be configured to receive a wireless interface device, such as a DB9 connector type which in some embodiments may be located on a ventilator or other patient monitor configured for patient care.

By way of example only, each of the connector ports 46, 50, 54, and 58 of the patient monitor 14 includes a plurality of inputs, e.g., sockets numbered 1 through 7, that are keyed or arranged in a pattern or scheme to ensure proper connection with its corresponding wired sensor or device. “Keying” may include, without limitation, any combination of structural features that are unique to that connector type, such as arrangement of sockets, the location of recesses or protrusions formed on the outer perimeter of the connector, or the location and shape of recesses or protrusions located on portions of the housing 30 that surround the connector. For example, connector 46 includes a notch 47 that is located at a different position that the notch 51 associated with connector 50. In addition, as indicated in FIG. 4, the connector ports 46, 50, 54, and 58 may be color-coded, e.g., yellow, gray, blue, or white, or otherwise labeled to ensure proper connection with its corresponding wired sensor or device. The color-coding may be related to how each port/connector is keyed. In other words, all ports/connectors of a particular color may be keyed the same.

A typical patient monitor 14 is typically adapted to be able to receive data from one type of wired physiological sensor (or a small subset of the available sensor types) through each of the wired sensor ports 46, 50, 54, and 58. For example, in patient monitor 14, the wired port 46 is only capable of interpreting and displaying data from a single wired end-tidal CO2 sensor.

Referring again to FIGS. 1 and 2, to upgrade the patient monitor 14 to provide wireless connectivity, one or more wireless interface devices 24 are provided. As best shown in FIG. 2, the wireless interface device 24 may take the form of a wireless dongle. The wireless interface device 24 is provided with a connector portion 25 arranged for physical insertion into one of the connector ports 46, 50, 54, and 58 of the wired sensor interface 38. For example, as best shown in FIG. 2, the wireless interface device 24 is shown physically inserted within the connector port 54, originally designed for use with a wired sensor. The wireless interface device 24 preferably receives power from the patient monitor 14 when physically inserted into one of the connector ports 46, 50, 54, and 58, and may include a voltage regulator to ensure a steady constant voltage supply received from the power source 36 through the connector ports. The voltage regulator may be desirable because voltages supplied to typical wired sensors can be as high as 20 volts. For example, the voltage regulator could be provided on the wireless interface device 24 to ensure a constant voltage of no more than 5 or, more preferably, 3.3 volts.

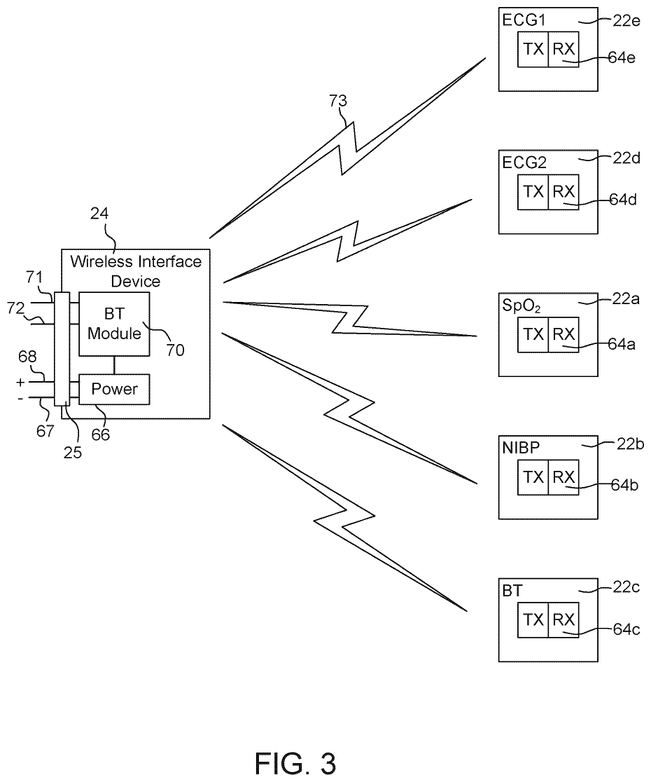

Referring now to FIGS. 1 and 3, in one embodiment, the wireless interface device 24 includes control circuitry configured to enable and/or implement wireless communication between the wireless interface device 24 and one or more wireless physiological sensors 22a-e that may be physically attached to the body of the patient 26. More specifically, a single wireless interface device 24 can be utilized to connect two or more, three or more, four or more, five or more, or ten or more wireless physiological sensors 22a-e. A wireless interface device 24 may be plugged into each available port on a given patient monitor, thus providing for an expanded capability for interconnecting wireless physiological sensors to a given patient monitor. In some embodiments, a single wireless interface device may be configured to plug into 2 ports at the same time, 3 ports at the same time, 4 ports at the same time, or more than 4 ports at the same time. A wireless interface device plugged into more than a single port is configured to increase bandwidth of the overall system, consume more data, and/or receive more physiological signals at the same time.

The wireless interface device 24 may be configured to receive wireless data signal transmissions from the wireless physiological sensors 22a-e and/or transmit wireless data signals (e.g., operational commands, parameters, and the like) that the wireless sensor devices are configured to receive and/or process. In the exemplary implementation shown in FIGS. 1 through 3, the wireless interface device 24 includes a power module 66 that is adapted to receive power from the connector port 54 through pins 67, 68 (also referred to herein as contacts) and Bluetooth module 70, which is adapted to transmit data to the patient monitor 14 via data I/O pins 71, 72. In alternate implementations, any suitable wireless communication interface could be utilized instead of the Bluetooth module 70. For example, an interface using communication protocols such as Wi-Fi, ZigBee, thread, infrared, or radio frequency (RF), or ANT could be used.

In some embodiments, the wireless communication interface is configured to send and/or receive data from only a single wireless sensor device. In other embodiments, the wireless communication interface is configured to toggle between data transfer from a set of wireless sensor devices. In some embodiments, toggling between data transfer from a set of wireless sensor devices comprises communicating with only a single wireless sensor device at a given time. In some embodiments, the wireless communication interface is configured to transmit a maximum of 2 MB, 3 MB, 1 MB, 5 MB, or 10 MB of data at a given time.

For example, in one embodiment, exemplary wireless physiological sensors 22a-e may include a plurality of wireless ECG sensor devices 22d-e attached to the chest of the patient 26; one or more wireless SpO2 physiological sensors 22a attached to a finger or wrist of the patient 26; one or more wireless body temperature (BT) physiological sensors 22c attached to an opposite wrist of the patient 26; and, one or more wireless non-invasive blood pressure (NIBP) physiological sensors 22b attached to the arm or leg of the patient 26. The foregoing sensor devices could be attached at other locations on the body of a patient 26, as is appropriate. For example, the wireless SpO2 sensor device 22a may be arranged for mounting on the finger of the patient 26 in order to detect blood-oxygen levels and/or pulse rate. In other embodiments, one or more of these same types of wireless sensor devices, or other types of wireless sensor devices could be attached to suitable locations on the body of the patient 26. Other types of wireless sensor devices include, for example, wireless end-tidal CO2 (etCO2) sensors, wireless pulse or heart rate (HR) sensors, wireless respiratory rate (RR) sensors, or wireless apnea detection sensors.

Each of the wireless physiological sensors 22a-e has a power source, e.g., one or more rechargeable or non-rechargeable batteries, and a transmitter/receiver 64a-e. The physiological signals of the patient 26 detected by the wireless sensors 22a-e may be transmitted from the wireless physiological sensors 22a-e to the wireless interface device 24 as sensor data signals through wireless signals 73, using any wireless communication protocol supported by the wireless interface device 24. By enabling wireless capability of the patient monitor 14 through the use of the wireless interface device 24, the patient monitor 14 may communicate with wireless sensors 22a-e, thus increasing the number and types of physiological parameters that can be monitored by the patient monitor 14, and increasing the utility of the limited number of connector ports 46, 50, 54, and 58. Under the present invention, the number and type of sensors and/or devices that can be employed is no longer limited by the number and/or type of ports disposed on the patient monitor.

In the event a single wireless interface device 24 provides insufficient bandwidth to support a desired number or variety of wireless sensors, one or more of the remaining connector ports 46, 50, and 58 may be made available for use with additional wireless interface devices 24 to assist in supporting the desired number of wireless sensors. The additional wireless interface devices 24 are preferably keyed to match the remaining connector ports 46, 50, and 58. In one embodiment, one or more wireless interface devices may be keyed to a particular connector port based upon the preset voltage for that connector port. Keying a wireless interface device to a particular connector port may include notching an inner ring connector, notching an outer ring connector, changing the rotational position of an inner ring connector with respect to an outer ring connector. The inner ring connector and outer ring connector may form part of connector portion 25 and the configuration thereof may be designed to mate specifically with one of ports 46, 50, 54, or 58 in the illustrated embodiment of FIG. 4. In another embodiment, where one or more sensor devices having high bandwidth requirements are utilized, a wireless interface device may be dedicated to those sensor devices. Alternatively, the remaining connector ports 46, 50, and 58 may be made available for connection with wired sensors.

In one embodiment, the wireless interface device 24 may be directly or indirectly coupled to the display/GUI 32 of the patient monitor 14, which may comprise an electronic display configured to visually display measurements and waveforms corresponding with the physiological data detected and transmitted by the wireless physiological sensors 22a-e.

In some implementations, embodiments of the present disclosure advantageously provide a solution for sending and receiving physiological data in the form of sensor data signals between the wireless physiological sensors 22a-e and the patient monitor 14 having connector ports originally designed for use with wired physiological sensors. Providing wireless functionality can improve safety and/or access to the patient 26 over other solutions that implement cables to connect devices within a healthcare environment, such as a hospital room, an operating room, or any other room or facility where a patient can receive medical care or treatment.

In implementations where the patient monitor 14 was initially programmed for use solely with wired physiological sensors, it may be necessary to reprogram the patient monitor 14 (for example, via a firmware update) to include the functionality necessary to support wireless physiological sensors connected to the patient monitor 14 through the wireless interface device 24. For example, the patient monitor 14 is preferably programmed to recognize when the wireless interface device 24 is connected to one of the ports 46, 50, 54, 58. In addition, the patient monitor 14 is preferably programmed to detect each wireless physiological sensor that is connected to the wireless interface device 24 and identify the wireless sensor type and/or input signal for each detected wireless physiological sensor. The wireless sensor type identification function could comprise a menu displayed by the patient monitor 14 that enables a user to select a wireless sensor type (e.g., from a pre-populated list). Alternatively, the wireless sensor type identification function could be performed either by (a) a code transmitted periodically by the detected wireless physiological sensor through the wireless interface device 24 or (b) from the nature of the signal received from the wireless physiological sensor (i.e., signal pattern recognition). In some embodiments, the received physiological signal may be encoded with a particular leading character corresponding to a particular sensor type.

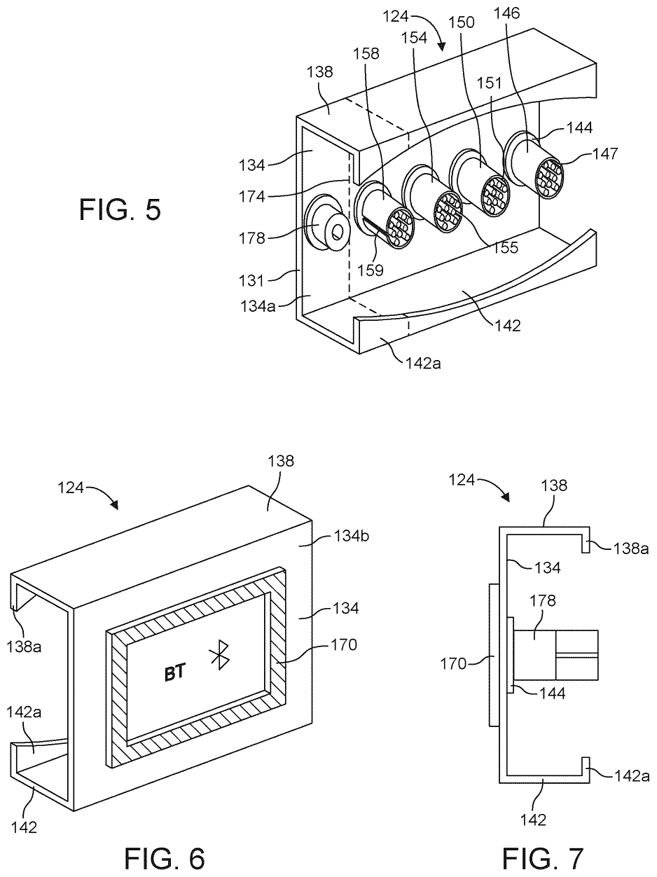

Referring now to FIGS. 5 through 7, there is shown another exemplary wireless interface device 124, which is arranged to connect to each available port 46, 50, 54, and 58 of the patient monitor 14 (FIG. 2). The wireless interface device 124 includes a clip 131 that houses the wireless interface (not shown), provides a mounting surface for connectors 146, 150, 154, and 158, and enables the wireless interface device to be removably engage the housing 30 of the patient monitor 14. In this illustrative example, the clip 131 forms a C-shape having a back panel 134, a top panel 138, and a bottom panel 142. The top and bottom panels 138, 142 may be integral with the back panel 134, and extend laterally from opposite ends of the back panel 134 to form the C-shape. The top panel 138 and the bottom panel 142 may include turned-in flanges 138a, 142a at their respective free ends, the turned-in flanges positioned to oppose one another to enable engagement with the housing 30 of the patient monitor 14. To secure the clip 131 to the patient monitor 14, the turned-in flange 138a is arranged to engage with the top surface of the patient monitor housing 30, and the turned in flange 142a is arranged to engage with the bottom surface of the patient monitor housing 30.

The clip 131 preferably is adapted to allow a power cable (not shown) to engage the port 78, which in some embodiments may be a non-invasive blood pressure (NIBP) connection port, of the patient monitor 14. The functionality may be provided by a serration 174 that facilitates trimming or cutting of the clip 131 to reduce its width, thereby providing access to the port 78. A cover 178 may be provided that covers and protects the port 78 when the serrated portion has not been removed, as well as providing additional stability to the clip 131. Alternatively, the clip 131 may include a cut-out portion (not shown) sized and located to provide through-access to the port 78.

A plurality of mounting bushings 144, e.g., four mounting bushings, extend from a rear surface 134a of the back panel 134 may be provided. The mounting bushings 144 are aligned with each of the connector ports 46, 50, 54, and 58 of the patient monitor 14. Mounted within each mounting bushing 144 is a connector 146, 150, 154, and 158. Each connector includes a plurality of electrical pins or contacts 160 including a plurality of data I/O contacts and a plurality of power contacts. Each connector is keyed to match its corresponding connector port 46, 50, 54, and 58. For example, as best shown in FIG. 5, each connector 146, 150, 154, and 158 includes a notch 147, 151, 155, 159 formed thereon, each notch being located at a rotational position that aligns with the port 46, 50, 54, or 58 engaged by each respective connector 146, 150, 154, and 158. As the C-shaped clip 131 is secured to the patient monitor 14, the connectors 146, 150, 154, and 158 physically and electrically connect to the connector ports 46, 50, 54, and 58. The wireless interface device 124 may receive power from the existing connector ports through the power contacts 160 of the connectors 146, 150, 154, and 158.

In some embodiments like that shown in FIGS. 6-7, the wireless interface device 124 is provided with a Bluetooth module 170 affixed to the front surface 134b of the back panel 134. Once the connectors 146, 150, 154, and 158 are physically and electrically connected to the connector ports 46, 50, 54, and 58, the Bluetooth module 170 is adapted to pair with the patient monitor and transmit data received from the wireless physiological sensors 22a-e to the patient monitor 14 through the connector ports 46, 50, 54, and 58. In alternate implementations, Bluetooth module 170 may be positioned in a different location on wireless interface device 124. In alternate implementations, any suitable wireless communication interface could be utilized instead of the Bluetooth module 170. For example, an interface using communication protocols such as Wi-Fi, ZigBee, thread, infrared, or radio frequency (RF), or ANT could be used.

As the wireless interface device 124 is configured to plug into all available ports of the patient monitor 14 simultaneously, it is configured to send and/or receive data from all of the various types of wireless physiological sensors 22a-e physically attached to the body of the patient 26. This includes the ECG sensor devices 22d-e attached to the chest of the patient 26; the one or more wireless SpO2 physiological sensors 22a attached to a finger or wrist of the patient 26; the one or more wireless body temperature (BT) physiological sensors 22c attached to an opposite wrist of the patient 26; and, the one or more wireless non-invasive blood pressure (NIBP) physiological sensors 22b attached to the arm or leg of the patient 26. The wireless interface device 124 could send and/or receive data from other types of wireless physiological sensors. The patient monitor 14 is preferably programmed to detect each wireless physiological sensor that is connected to the wireless interface device 124 and identify the wireless sensor type and/or input signal for each detected wireless physiological sensor.

As the wireless interface device 124 may be plugged into all of the available ports on the patient monitor 14, it provides expanded capability for interconnecting wireless physiological sensors to a given patient monitor. In this manner, the wireless interface device 124 provides increased bandwidth to the overall system, consumes more data, and/or receives more physiological signals from a greater number and different types of physiological sensors 22a-e at the same time.

As discussed above, the wireless interface device 124 shown in FIGS. 5-7 is configured to plug into the four available connector ports 46, 50, 54, and 58 of the patient monitor 14 (FIG. 2). In alternative embodiments, where it is desired to wirelessly interface with a fewer or a greater number of connector ports, e.g., two ports, three ports, or more than four ports, the interface device may be provided with a fewer or greater number of connectors. Wireless interface device 124 may be selected with two ports, three ports, four ports, or more than four ports in order to match the number of available connector ports on the patient monitor. The wireless interface device 124 could be configured to provide a plurality of connectors, but fewer than the number of connector ports provided with the patient monitor. Similarly, the wireless interface device 124 could also be configured to leave one or more connector ports exposed (and not provide a corresponding connector) for connectors ports that are for sensor types for which it may be desirable to have a wired connection.

Conventionally, a connector port on a monitoring device such as a patient monitor supports only one parameter that is assigned to the port from a single sensor. As described further herein, in some embodiments, a connector port can support multiple parameters concurrently from multiple sensors. In some implementations, an interface device may be physically inserted into a single connector port of a patient monitor and may be configured to receive one or more physiological parameters concurrently from multiple sensors. In this manner, a single connector port of a patient monitor can support one or more parameters from one or more wired sensors and/or one or more wireless sensors.

More particularly, multi-parameter support (e.g., SpO2 and EtCO2) and/or multi-sensor support (e.g., dual SpO2) is provided on a single connector port of a monitoring device. When a connector port is configured to receive data from a plurality of sensors (i.e., two or more sensors), the data is distinguishable as to the type of data or parameter being received and attributable to the appropriate sensor that is providing that data, regardless of whether that sensor is a wired sensor or a wireless sensor. In some implementations, one or more bit flags may be used in the data packets received from each of the sensors (e.g., in the protocol packet headers) to identify the type of data (i.e., the parameter being measured) and/or the sensor that is providing the data. In this manner, the monitoring device that receives the data via the connector port can accurately and efficiently process the data for storage, usage, further processing, and/or output such as display or other indication or notification.

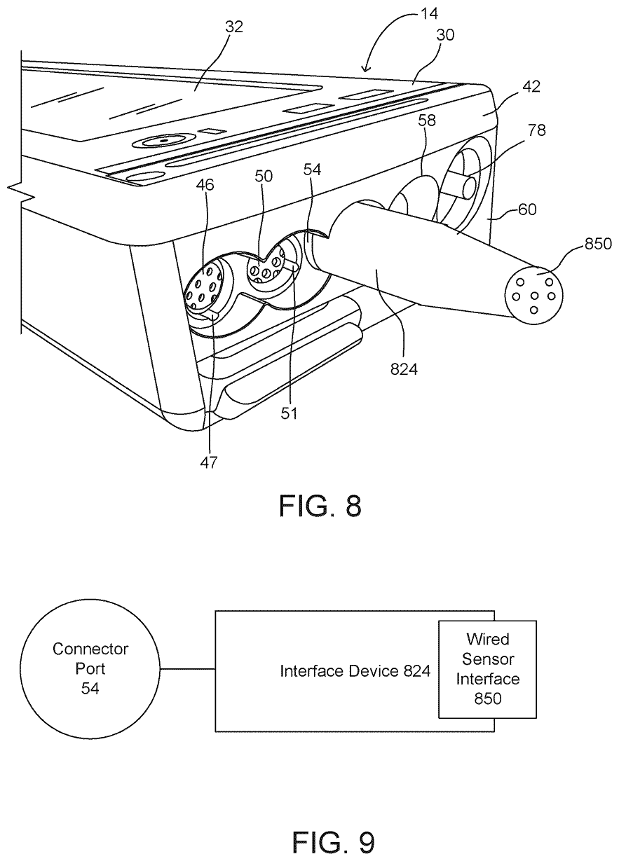

FIGS. 8 and 9 show the patient monitor 14 and an interface device 824 adapted to physically and electrically connect to the connector port 54 of the patient monitor 14. The interface device includes a wired sensor interface port 850, which is adapted to provide support for a different physiological parameter than connector port 54 was originally designed to support. For example, the connector port 54 (e.g., a digital port) that is adapted to support an EtCO2 sensor could be enhanced to enable support for a second SpO2 sensor (the first one being plugged into another connector port of the patient monitor that is adapted for an SpO2 sensor). In an implementation, for example, the interface device 824 may have a EtCO2 male connector on one end (the end that connects to the connector port 54) and the wired sensor interface 850 consists of an SpO2 female connector, which enables a SpO2 wired sensor device to connect through the connector port 54.

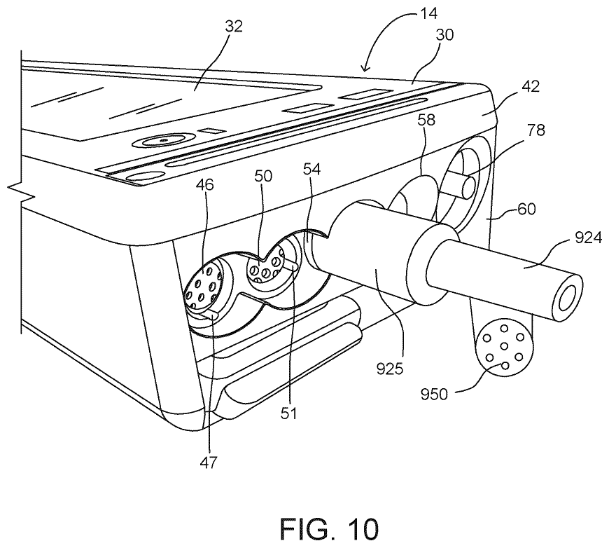

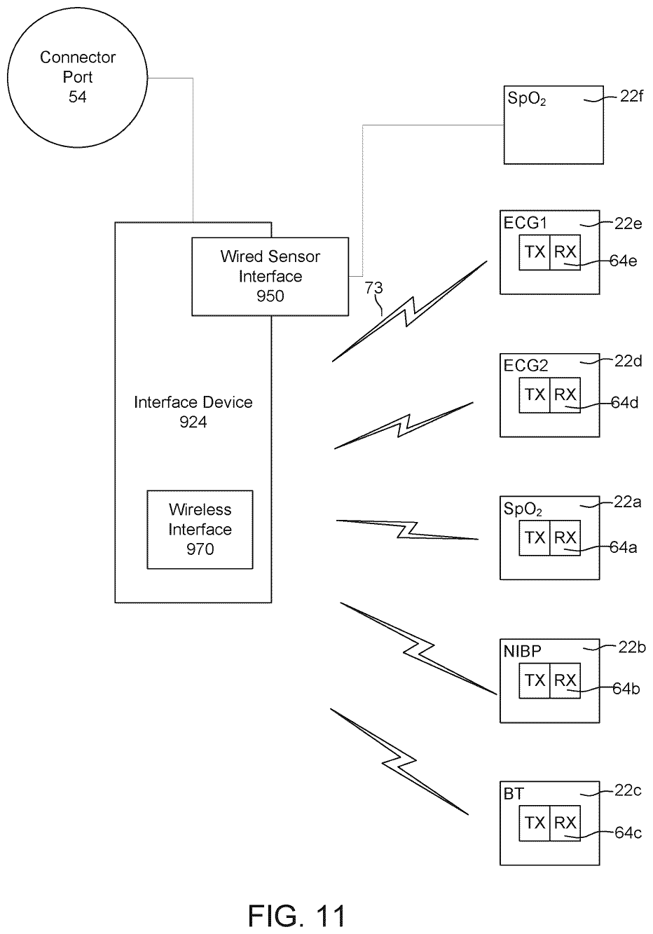

Referring to FIGS. 10 and 11, in some exemplary implementations, the interface device 924 may be adapted to be capable of connecting to both wired and wireless physiological sensors. The interface device 924 has the same wireless functionality as interface device 24, described above, which enables it to send and/or receive data from all of the various types of wireless physiological sensors 22a-e physically attached to the body of the patient 26. The components providing wireless functionality are represented schematically in FIG. 11 as a wireless interface 970. Similar to the interface device 824 of FIGS. 8 and 9, the interface device 924 also includes a wired sensor interface port 950, which is adapted to provide support for a wired physiological sensor (for example, the wired SpO2 sensor 22f shown in FIG. 11). The wired physiological sensor interface port 950 may be adapted to connect to a sensor type that is either the same as or different from the physiological parameter than connector port 54 was originally designed to support 10.

In some implementations, the interface device 924 may have a smart capability with a microprocessor unit that supports wireless technology, such as Bluetooth® low energy. This enhances its capability to receive wireless physiological data from one or more wireless sensors. Software updates to the patient monitor could be used to map received data to expected parameter data. This can be accomplished, for example, by adding bit flags to the protocol headers of the data received from the physiological sensors.

The interface device 950 provides functionality that enhances connectivity options for each connection port 54 of the patient monitor 14. The interface device 950 enables two sensors for the same physiological parameter to be connected through a single connector port of the patient monitor 14. For example, the interface device 950 enables data from both a wireless and a wired SpO2 sensor could be received by the connector port 54. Similarly, the interface device 950 enables sensors of two different physiological parameters to be connected through a single connector port of the patient monitor 14. For example, the interface device 950 enables data from both a wireless EtC02 sensor and a wireless SpO2 sensor to be received by the connector port 54.

Although only one wired sensor interface 950 and one wireless interface device 924 is shown in FIGS. 10 and 11, this is not intended to be limiting as any number of wired sensor interfaces and wireless interface devices, and thus wired sensors and wireless sensors, may be supported and used depending on the implementation.

While various embodiments have been disclosed, it will be apparent to those skilled in the art that various changes and modifications can be made which will achieve some of the advantages of the concepts disclosed herein without departing from the spirit and scope of the present disclosure. It will be obvious to those reasonably skilled in the art that other components performing the same functions may be suitably substituted. It is to be understood that other embodiments may be utilized and structural or logical changes may be made without departing from the scope of the present disclosure. It should be mentioned that features explained with reference to a specific figure may be combined with features of other figures, even in those not explicitly mentioned. Such modifications to the general inventive concept are intended to be covered by the appended claims and their legal equivalents.

Furthermore, the following claims are hereby incorporated into the detailed description, where each claim may stand on its own as a separate example embodiment. While each claim may stand on its own as a separate example embodiment, it is to be noted that, although a dependent claim may refer in the claims to a specific combination with one or more other claims, other exemplary embodiments may also include a combination of the dependent claim with the subject matter of each other dependent or independent claim. Such combinations are proposed herein unless it is stated that a specific combination is not intended. Furthermore, it is intended to include all features of a claim that depend to any other independent claim even if this claim is not directly made dependent to the independent claim.

It is further to be noted that methods disclosed in the specification or in the claims may be implemented by a device having means for performing each of the respective acts of these methods. For example, the techniques described in this disclosure may be implemented, at least in part, in hardware, software, firmware, or any combination thereof. For example, various aspects of the described techniques may be implemented within one or more processors, including one or more microprocessors, DSPs, ASICs, or any other equivalent integrated or discrete logic circuitry, as well as any combinations of such components.

Further, it is to be understood that the disclosure of multiple acts or functions disclosed in the specification or in the claims may not be construed as to be within the specific order. Therefore, the disclosure of multiple acts or functions will not limit these to a particular order unless such acts or functions are not interchangeable for technical reasons. Furthermore, in some embodiments a single act may include or may be broken into multiple sub acts. Such sub acts may be included and part of the disclosure of this single act unless explicitly excluded.

It is to be understood that the embodiments described herein are merely exemplary and that a person skilled in the art may make many variations and modifications without departing from the spirit and scope of the invention. All such variations and modifications are intended to be included within the scope of the invention, as defined by the following claims.

1-8. (canceled)

9. A wireless interface device for use with a patient monitor having at least one wired physiological sensor port, each at least one wired physiological sensor connector port having a plurality of physiological sensor port contacts, the wireless interface device comprising:
at least one connector, each at least one connector comprising a plurality of electrical contacts including a plurality of data I/O contacts and a plurality of power contacts, and a power module in electrical communication with the plurality of power contacts; and
a wireless communication module in electrical contact with the data I/O contacts;
wherein the at least one connector is shaped to be physically connected to one of the at least one wired physiological sensor ports and to enable each of the plurality of connector electrical contacts to be electrically connected with one of the plurality of physiological sensor port contacts when the at least one connector is physically connected to the at least one wired physiological sensor port;
wherein the wireless communication module is adapted to establish a wireless data connection with at least one wireless physiological sensor; and
wherein when the at least one connector is connected to one of the at least one wired physiological sensor port, the wireless communication module is adapted to pair with the patient monitor and transmit received data from the at least one wireless physiological sensor through at least one of the at least one wired physiological sensor port.

10-11. (canceled)

12. The wireless interface of claim 9, wherein the wireless interface device is adapted to connect to two or more of the ports of the patient monitor.

13. (canceled)

14. The wireless interface device of claim 9, wherein the at least one wired physiological sensor port comprises a plurality of wired physiological sensor ports, the at least one connector of the wireless interface device comprises a plurality of connectors, and the plurality of connectors being equal or less in number than the plurality of wired physiological sensor ports.

15-16. (canceled)

17. The wireless interface device of claim 14, further comprising a housing to which the plurality of connectors, the power module, and the wireless communication module are attached, wherein the housing is adapted to removably engage a patient monitor.

18. The wireless interface device of claim 17, wherein the housing is c-shaped.

19. The wireless interface device of claim 17, wherein the housing is adapted allow a power port of a patient monitor to remain exposed when each of the plurality of connectors is mechanically and electrically engaged to a respective one of the plurality of wired physiological sensor ports.

20. The wireless interface device of claim 19, wherein the housing comprises a serration that enables a first portion of the housing covering to be separated from the housing, wherein the first portion covers the power port when each of the plurality of connectors is mechanically and electrically engaged to a respective one of the plurality of wired physiological sensor ports.

21. The wireless interface device of claim 9, wherein the wireless interface device is cylindrically shaped.

22. A physiological monitoring system, comprising:
a patient monitor comprising:
at least one processor, memory, a wireless communication interface adapted to communicate with a network, a graphical user interface, a power source, and a plurality of sensor ports, the plurality of sensor ports comprising a first sensor port;
wherein the at least one processor is adapted to detect whether a wired physiological sensor or a wireless interface device is connected to the first sensor port, the wired physiological sensor being adapted to sense a first physiological parameter, the wireless interface device being adapted to connect with a first wireless physiological sensor adapted to sense a second physiological parameter;
wherein when the wired physiological sensor is detected as being connected to the first sensor port, the at least one processor is adapted to display on the graphical user interface data representative of the physiological parameter based on data received from the wired physiological sensor; and
wherein when the wireless interface device is detected as being connected to the first sensor port, the at least one processor is adapted to display on the graphical user interface data representative of the second physiological parameter based on data received from a wireless the wireless physiological sensor; and
a wireless interface device for use with the patient monitor, the wireless interface connector comprising:
at least one connector, each at least one connector comprising a plurality of electrical contacts including a plurality of data I/O contacts and a plurality of power contacts, and a power module in electrical communication with the plurality of power contacts; and
a wireless communication module in electrical contact with the data I/O contacts.

23. The physiological monitoring system of claim 22, wherein the wireless interface device comprises a C-shaped housing adapted to removably engage a patient monitor.

24. The physiological monitoring system of claim 22, wherein the processor is further adapted to detect a wireless communication connection between the wireless interface device and at least one wireless physiological sensor.

25. (canceled)

26. The physiological monitoring system of claim 23, wherein the C-shaped housing includes a serration that permitting a first portion of the housing to be separated from the housing.

27. The physiological monitoring system of claim 24, wherein the wireless interface device is adapted to connect to a patient monitor by two or more of the sensor ports.

28. The physiological monitoring system of claim 22, wherein the wireless interface device is cylindrically shaped.

29-37. (canceled)

38. An interface device for a first physiological sensor port of a patient monitor, the first physiological sensor port being adapted to receive data representative of a first physiological parameter from a wired physiological sensor adapter, the interface comprising:
a patient monitor interface connector adapted to electrically and mechanically connect with the first physiological sensor port; and
a physiological sensor interface port adapted to electrically and mechanically connect with a wired physiological sensor adapted to provide a signal representative of a second physiological parameter, the second physiological parameter being different from the first physiological parameter.

39. The interface device of claim 38, wherein the interface device is cylindrically-shaped.

40. The interface device of claim 38, wherein the physiological sensor interface port is keyed differently from the first physiological sensor port.

41. The interface device of claim 38, wherein the patient monitor interface connector is one of a plurality of patient monitor interface connectors and the interface device is adapted to connect to a patient monitor by two or more ports.

42. The interface device of claim 38, comprising a C-shaped housing adapted to removably engage a patient monitor.

43. The interface device of claim 42, wherein the C-shaped housing includes a serration that permitting a first portion of the housing to be separated from the housing.

44-47. (canceled)
